(19) United States
(12) Patent Application Publication
(10) Pub. No.: US 2019/0358182 A1
(43) Pub. Date: Nov. 28, 2019

(54) NOVEL MECHANISM FOR DECREASING OPIOID REWARD

(71) Applicant: THE TRUSTEES OF INDIANA UNIVERSITY, Bloomington, IN (US)

(72) Inventor: Andrea Grace HOHMANN, Bloomington, IN (US)

(21) Appl. No.: 16/420,972

(22) Filed: May 23, 2019

Related U.S. Application Data
(60) Provisional application No. 62/696,738, filed on Jul. 11, 2018.
(60) Provisional application No. 62/675,944, filed on May 24, 2018.

(51) Int. Cl. A61K 31/192; A61P 25/36; A61K 31/4192

(52) U.S. Cl. CPC A61K 31/192; A61K 9/0019; A61K 31/4192; A61P 25/36

(57) ABSTRACT

The present disclosure provides compositions and methods for treating patients suffering from opioid dependency and/or addiction wherein the patient is administered a pharmaceutical composition that disrupts the protein-protein interface between nNOS and PSD95.

### CROSS REFERENCE TO RELATED APPLICATIONS

This application claims priority to the following: U.S. Provisional Patent Application No. 62/696,738, filed on Jul. 11, 2018, and U.S. Provisional Patent Application No. 62/675,944 filed on May 24, 2018. The disclosure of each application is hereby expressly incorporated by reference in its entirety.

### GOVERNMENT RIGHTS

This invention was made with government support under DA042584 awarded by the National Institutes of Health. The government has certain rights in the invention.

### BACKGROUND

Opioids have been used for centuries for chronic pain treatment, but their utilities are limited by potential for abuse. In the United States, unintentional deaths due to prescription drug overdoses have more than tripled since 1990, more than deaths attributed to cocaine and heroin combined. The increase in unintentional drug overdose death rates has mainly been driven by increased use of opioid analgesics. Inadequate treatment for pain, exacerbated by incomplete analgesic efficacy and narcotic abuse liability, contributes to escalating drug use, resulting in socioeconomic costs estimated at $600 billion annually. Improving the safety, efficacy, side effect profile and abuse liability of opioid analgesics thus remains an urgent medical need.

Opioid reward and dependence require the excitatory neuro transmitter glutamate. Excessive glutamate signaling through N-methyl-D-aspartate receptors (NMDARs) is implicated in altered forms of neuronal plasticity associated with opioid reward and dependence. Excessive NMDAR stimulation triggers a signaling cascade involving activation of the enzyme neuronal nitric oxide synthase (nNOS), which catalyzes formation of the signaling molecule nitric oxide (NO), which promotes addition-related behaviors.

Current treatment of opioid abuse relies on pharmacological approaches (methadone or buprenorphine) or behavioral therapies, both of which have significant limitations. Inhibition of aberrant glutamatergic hyperexcitability and inhibition of nNOS reduces addition-related behaviors in preclinical studies. However the therapeutic potential of NMDA receptor antagonists and NOS inhibitors are limited by severe side effects. Accordingly better approaches to dealing with the factors contributing to opioid abuse (reward and dependence) are needed, including an alternative method of inhibiting aberrant glutamatergic hyperexcitability. As disclosed herein applicant proposes disruption of a key signaling pathway involved in opioid reward as a therapeutic strategy to bypass and prevent narcotic abuse liability.

### SUMMARY

The present disclosure provides a method for decreasing opioid abuse liability by inhibiting the development of opioid reward and escalating drug use associated with opioid analgesics. The novel approach disclosed herein utilizes small molecules to functionally decouple signaling complexes downstream of NMDAR activation to eliminate aberrant NMDAR-dependent nitric oxide signaling. In one embodiment, the present disclosure provides a method for functionally decoupling NMDARs from nNOS signaling to inhibit opioid reward without one or more unwanted side effects of global NMDAR antagonists or nonselective NOS catalytic inhibitors. In one embodiment the method comprises disrupting the protein-protein interface between nNOS and postsynaptic density 95 kDA (PSD95), wherein PSD95 is a scaffolding protein which tethers nNOS to NMDARS.

In accordance with one embodiment compositions and methods are provided for treating patients suffering from opioid dependency and/or addiction wherein the patient is administered a pharmaceutical composition that disrupts the protein-protein interface between nNOS and PSD95. In another embodiment a model system and method is provided for analyzing patient responses to opioids and addiction.

### BRIEF DESCRIPTION OF THE DRAWINGS

FIG. 1 shows PSD95-nNOS inhibitors disrupt NMDAR signaling, providing a model for disrupting morphine-induced reward, relapse, tolerance and dependence without side effects of NMDAR antagonists. Bold lines show blockade, arrows show activation.

FIG. 2 shows IC87201 and ZL006 disrupt PSD95-nNOS but not unrelated PSD95-ERB4 interactions in Alphascreen.

FIGS. 3A-3C present data from a conditioned place preference study where one chamber was paired with the drug treatment and the other chamber was paired with the vehicle. Animals are subsequently tested in the drug free state to determine if the drug pairing produced reward or aversion in the animals. FIGS. 3A-C shows that single trial morphine-induced CPP (6 mg/kg i.p.) (p<0.02 vs. vehicle-paired chamber) is blocked in rats by acute treatment with either ZL006 or IC87201. Naïve animals exhibit robust single trial CPP to morphine (FIG. 3A) whereas acute treatment with ZL006 or IC87201 failed to do so (data not shown). However, single trial morphine-induced CPP was notably absent when the opioid was co-administered with either ZL006 or IC87201 (FIG. 3B and FIG. 3C, respectively).

FIGS. 4A-4F are graphs presenting data that extends the CPP observations to repeated pairings with each drug treatment using a 12 day conditioned place preference protocol. FIGS. 4A & B show that four repeated pairings with either ZL006 (10 mg/kg i.p.×4 pairings on alternate days; see FIG. 4A) or IC87201 (10 mg/kg i.p.×4 pairings on alternate days; see FIG. 4B) did not produce either conditioned place preference or aversion when administered alone. These observations suggest that neither ZL006 nor IC87201, was associated with either reward or aversion in the absence of morphine. By contrast, repeated pairings with morphine (6 or 10 mg/kg i.p.×4 pairings) produced robust conditioned place preference under identical conditions (FIGS. 4C & D). Moreover, when ZL006 (10 mg/kg i.p.×4 pairings) or IC87201 (10 mg/kg i.p.×4 pairings) was coadministered with morphine (6 mg/kg i.p.), conditioned place preference to morphine was no longer observed (FIGS. 4E & F). These observations indicate that two structurally distinct nNOS-PSD95 inhibitors block opioid reward in a conditioned place preference assay, even under stringent conditions of testing where drug pairings are repeated 4 times and animals are tested in the drug free state.

FIGS. 5A & 5B are graphs presenting data demonstrating that IC87201 and ZL006 do not impair memory in the Morris Water maze (FIG. 5A; see Smith et al. (2016) Behavioural Brain Research 305: 23-29 for memory experiments. In this manuscript, using cell culture we also show that PSD95-nNOS inhibitors suppress cGMP levels, a marker of NO formation) or impair motor performance in the rota-rod test (FIG. 5B) Memory for source (i.e. origin) of information is selectively vulnerable to impairment by NMDAR antagonist (MK801 0.1 or 0.2 mg/kg i.p.) under conditions in which spatial working memory is spared. By contrast, PSD95-nNOS inhibitors (IC87201 and ZL006; 10 mg/kg i.p.) did not impair any memory function under identical testing conditions. These observations suggest that PSD95-nNOS inhibitors do not produce memory impairment characteristic of NMDAR antagonists, and that failure to remember the chamber paired with PSD95-nNOS inhibitor cannot confound our interpretation of the conditioned place preference experiments described in FIGS. 3A-3C and 4A-4F. Rats were trained in a radial arm maze task that tests both source and spatial working memory for these experiments. Data are average of three trials 30 min after drug injection (n=6/group).

FIGS. 6A-6C show Naïve, sham and neuropathic rats self-administer morphine (0.1 mg/kg/injection) on FR1 schedule. Dose did not alter mechanical thresholds in naïve rats (See FIG. 6A). Active lever presses were greater than inactive presses in all groups (***P<0.001). Neuropathic rats responded (FR1) preferentially on active levers (previously morphine-paired; FIG. 6B) but not inactive lever (FIG. 6C) during extinction (initial switch from morphine to vehicle self-administration).

FIGS. 7A-7D. shows that as expected morphine produces robust dopamine efflux following electrical stimulation of the medial forebrain bundle (FIG. 7A). By contrast, ZL006, by itself, does not increase dopamine (DA) efflux under conditions in which morphine (FIG. 7A) induces robust DA efflux. These observations suggest that ZL0005 by itself is unlikely to produce reward. Note that a robust increase in evoked DA was seen following morphine (6 mg/kg i.p.) administration in rats pre-treated with vehicle. ZL006 (10 mg/kg i.p.) treatment does not elicit DA efflux (FIG. 7B), does not affect the amount of DA released per stimulus pulse (FIG. 7C), and does not affect the amount of DA uptake per stimulus pulse FIG. 7D). Vehicle: 20% dimethyl sulfoxide with 80% ethanol:emulphor:saline in a 1:1:8 ratio. (n=6 per group).

FIGS. 8A-8C show electrically evoked DA recorded in NAc in an anesthetized rat (FIG. 8A) recorded following i.p. injection of vehicle, ZL006 (10 mg/kg i.p.), morphine (6 mg/kg i.p.) and ZL006 (10 mg/kg i.p.) plus morphine (6 mg/kg i.p.). Voltammogram shows measurement of DA. Evoked DA response before (upper trace) and 30 min after (lower trace) 10 mg/kg i.p. ZL006 administration (FIG. 8B), demonstrating ZL006-induced decrease in DA evoked response. Arrow shows MPH stimulation. Each point is a DA signal recorded every 100 ms. Post-calibration testing ruled out a loss of electrode sensitivity. Inset of FIG. 8B: Voltammograms recorded at before and after peaks. FIG. 8C shows group data (n=4-6 per group) demonstrating that ZL006 (10 mg/kg i.p.) suppresses morphine-induced dopamine efflux in nucleus accumbens shell. Note that morphine-induced dopamine efflux is blunted in rats that received ZL006 (10 mg/kg i.p.) co-administered with morphine (6 mg/kg i.p.). ZL006 alone does not increase DA efflux but blocks morphine-induced dopamine efflux in these studies (FIG. 8C).

### DETAILED DESCRIPTION

### Definitions

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviating the symptoms associated with a specific disorder or condition and/or preventing or eliminating said symptoms. For example, treating addiction includes preventing or inhibiting the reward and motivation for taking the addictive substance.

As used herein an “effective” amount or a “therapeutically effective amount” of a small molecule therapeutic agent refers to a nontoxic but sufficient amount of the therapeutic agent to provide the desired effect. For example one desired effect would be the prevention aberrant NMDAR-dependent nitric oxide signaling, as measured, for example, by a decrease in NO signaling.

As used herein the term “drug dependency” defines a state wherein a patient experiences symptoms of withdrawal when administration of the drug is disrupted. An individual having a drug dependency is not necessarily addicted to the drug.

As used herein the term “drug addiction” defines a state of compulsive need for the drug despite negative consequences associated with taking the drug.

### EMBODIMENTS

NMDAR-dependent nNOS signaling is important for induction and maintenance of addiction (FIG. 1). NMDARs form a multiprotein signaling complex, whose activation increases nNOS activity and produces NO. Excessive NMDAR stimulation triggers a signaling cascade involving activation of the enzyme neuronal nitric oxide synthase (nNOS), which catalyzes formation of the signaling molecule nitric oxide (NO), which promotes addiction-related behaviors. More particularly, NMDAR activation results in Ca influx and nNOS activation via an intermediary (the scaffolding protein postsynaptic density 95 kDa (PSD95)) which tethers nNOS to NMDAR.

Because NMDAR-dependent activation of nNOS requires PSD95, applicant has targeted the disruption of PSD95nNOS protein-protein interactions as a mechanism for decreasing opioid abuse liability (FIG. 1). This approach targets pathways downstream of NMDAR but upstream of the nNOS catalytic domain and should result in a more favorable safety profile compared to NMDAR antagonists or nonselective NOS inhibitors. NMDARs colocalize with and exhibit bidirectional crosstalk with MORs, the target of morphine. Interruption of NMDAR-dependent NO signaling does not impede MOR antinociception, but rather, attenuates development of morphine tolerance. Our previous studies demonstrate that inhibitors of the PSD95-nNOS protein-protein interface (e.g., IC87201 and ZL006) suppress pain hypersensitivity in chronic pain models without altering nociceptive responding in naïve animals (Neuropharmacology (2015); 97:464-75). Applicant has now discovered that PSD95-nNOS disruption blocks opioid reward using both preclinical animal models and Fast-scan cyclic voltammetry (FSCV), wherein in vivo release and uptake of DA in NAc, a key component of the reward circuit is blocked. Furthermore, applicants anticipate that NMDAR activation of DA neurons may underlie drug seeking and administration of inhibitors of the PSD95-nNOS protein-protein interface could decrease drug seeking behaviors.

Inhibition of aberrant glutamatergic hyperexcitability and inhibition of nNOS have been reported to reduce addiction-related behaviors in preclinical studies. However, the therapeutic potential of NMDA receptor antagonists and NOS inhibitors are limited by severe adverse side effects associated with their use. Specifically, clinical use of NMDAR antagonists is reported to be associated with adverse side effects including memory and motor impairment and hallucinations. These limitations fostered research efforts targeting signaling pathways downstream of NMDAR by developing nNOS inhibitors. However, nNOS inhibitors are not selective for nNOS but inhibit other NOS isoforms (e.g. endothelial NOS), which translates into adverse side effects (e.g. increased blood pressure, hypertension, pancreatitis. Thus, despite, elucidation of the critical role of NMDAR-dependent nNOS signaling in opioid reward and dependence, this information has not translated into better pharmacotherapies for pain in humans.

Targeted disruption of specific protein-protein interactions offers potential for maximizing therapeutic efficacy with limited side effects based upon its capacity to selectively eliminate aberrant signaling cascades while retaining those required for normal physiological function. A peptide that disrupts NMDAR-PSD95 interaction shows efficacy in phase II clinical trials in patients undergoing cerebral aneurysm repair. Disrupting the NMDAR-PSD95 interaction, however, is likely to produce more undesirable side effects than blocking the PSD95-nNOS interaction selectively.

Accordingly, as disclosed herein, applicants have targeted a specific protein-protein interaction in the NMDAR signaling complex to eliminate NMDAR-dependent NO signaling and block opioid reward, and validate a new class of chemical compounds distinct from traditional enzyme catalytic inhibitors or receptor antagonists for reducing opioid abuse liability. Small molecules offer advantages over peptides, which are rapidly degraded and not suitable for chronic use. The present disclosure represents a novel way to disrupt NMDAR-nNOS signaling to lessen opioid reward, and therein, potentially abuse liability, without unwanted side effects of NMDAR antagonists or nonselective NOS inhibitors. Moreover, because PSD95-nNOS inhibitors suppress pain hypersensitivity, combining opioids with PSD95-nNOS inhibitors could improve opioid therapeutic ratios by enhancing antihyperalgesic efficacy while reducing unwanted side effects in chronic pain patients. Our use of FSCV, moreover, provides a first-ever assessment of PSD95-nNOS disrupters on in vivo DA signaling in the forebrain reward circuit, which becomes critical information for attempts to decrease drug craving.

NMDAR-dependent activation of nNOS requires a scaffolding protein (postsynaptic density 95 k Da (PSD95) that tethers nNOS to the NMDAR signaling complex. NMDAR activation also promotes the participation of dopamine (DA), a key driver of drug reward. As disclosed herein, disruption of protein-protein interactions between PSD95 and nNOS will inhibit NMDAR-dependent NO signaling and thus interferes with DA signaling of opioid reward.

The present disclosure is directed to methods of disrupting PSD95-nNOS protein-protein interactions to suppress opioid-induced dopamine efflux in a neural cell, wherein the molecule does not itself produce a reward response in neural cells of a patient. Disruption of PSD95-nNOS interactions is anticipated to attenuate opioid-induced dopamine dynamics in the nucleus accumbens shell, a key component of the reward circuit. Disruption of the PSD95-nNOS interface will be used to eliminate opioid reward, while retaining therapeutic efficacy, while advantageously also bypassing unwanted side effects of associated with the administration of both NMDAR antagonists and NOS catalytic inhibitors. In accordance with the present invention, PSD95-nNOS inhibitors are administered to patients to reduce opioid reward, and provide a treatment for opioid addiction and dependency, thus filling a major gap in an area of unmet therapeutic need.

As disclosed herein, a nNOS-PSD95 disruptor compound is provided wherein the compound inhibits opioid-induced dopamine efflux in a neural cell, and wherein the molecule does not itself produce a reward response in the neural cell. In one embodiment, the compound further blocks opioid reward responses in a neural cell. In one embodiment, the compound is ZL006 or bioactive equivalent derivative thereof. Alternatively, in one embodiment the compound is IC87201 or bioactive equivalent derivative thereof.

In accordance with one embodiment, a method of blocking opioid-reward responses in a neural cell is provided wherein the cell is contacted with a composition comprising a nNOS-PSD95 disruptor molecule (i.e., a compound that disrupts the protein-protein interface between nNOS and PSD95). In one embodiment the method relates to blocking the opioid-reward response induced by an opioid selected from the group consisting of oxycodone, oxymorphone, hydromorphone, hydrocodone, morphine, tapentadol, tramadol, buprenorphine, and the physiologically acceptable salts thereof, and combinations thereof.

In accordance with one embodiment, a method of inhibiting opioid-induced dopamine efflux in a neural cell is provided wherein the cell is contacted with a PSD95-nNOS inhibitor. In one embodiment, the PSD95-nNOS inhibitor is a compound that disrupts the protein-protein interface between nNOS and PSD95, and in one embodiment the PSD95-nNOS inhibitor is a small molecule. In one embodiment the method relates to inhibiting opioid-induced dopamine efflux induced by an opioid selected from the group consisting of oxycodone, oxymorphone, hydromorphone, hydrocodone, morphine, tapentadol, tramadol, buprenorphine, and the physiologically acceptable salts thereof, and combinations thereof.

In accordance with one embodiment small molecule inhibitors of PSD95-nNOS protein-protein interactions are used to block opioid reward, including an attenuation of DA transmission, while circumventing the unwanted side effects of associated with the administration of NMDAR antagonists.

In accordance with one embodiment a method of inhibiting opioid-induced dopamine efflux in a neural cell is provided wherein the cell is contacted with a compound comprising the general structure of Formula I:

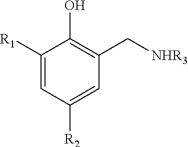

wherein R₁ and R₂ are independently halo, optionally wherein R₁ and R₂ are independently selected from the group consisting of I, F, Br and Cl; and R₃ is aryl, optionally a 6 member or 9 member aryl. In one embodiment R₃ is phenyl. In one embodiment, R₃ is

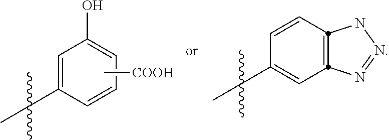

In accordance with one embodiment a method of inhibiting opioid-induced dopamine efflux in a neural cell is provided wherein the cell is contacted with a compound comprising the general structure of Formula II:

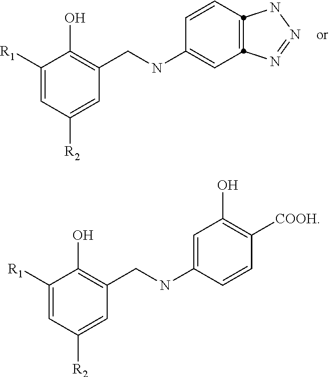

wherein R₁ and R₂ are independently selected from F, Br and Cl. In one embodiment R₁ and R₂ are independently selected from F and Cl and in one embodiment R₁ and R₂ are each Cl.

In accordance with one embodiment, a method of treating a patient with an opioid addition is provided wherein the patient is administered a pharmaceutical composition comprising a PSD95-nNOS inhibitor. In one embodiment the PSD95-nNOS inhibitor is a compound that disrupts the protein-protein interface between nNOS and PSD95, and in one embodiment the PSD95-nNOS inhibitor is a small molecule.

The PSD95-nNOS inhibitor can be administered to a patient using any standard route of administration, including parenterally, such as intravenously, intraperitoneally, subcutaneously or intramuscularly, intrathecally, transdermally, rectally, orally, nasally or by inhalation. In one embodiment, the composition is administered subcutaneously or intramuscularly.

In accordance with one embodiment a method of treating a patient with an opioid addition is provided wherein the patient is administered a pharmaceutical composition comprising a compound of the general structure of Formula I:

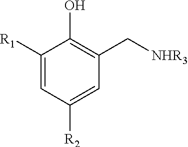

wherein R₁ and R₂ are independently halo, optionally wherein R₁ and R₂ are independently selected from the group consisting of I, F, Br and Cl; and R₃ is aryl, optionally a 6 member or 9 member aryl. In one embodiment R₃ is phenyl. In one embodiment, R₃ is

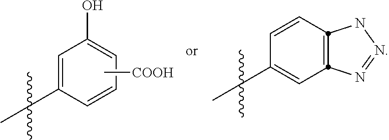

In accordance with one embodiment a method of treating a patient with an opioid addition is provided wherein the patient is administered a pharmaceutical composition comprising a compound having the general structure of Formula II:

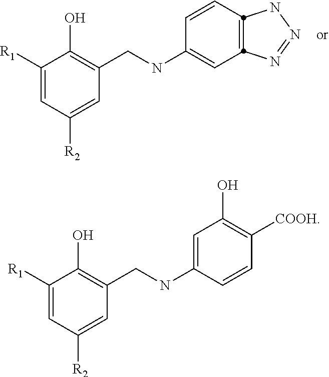

wherein R₁ and R₂ are independently selected from F, Br and Cl. In one embodiment R₁ and R₂ are independently selected from F and Cl and in one embodiment R₁ and R₂ are each Cl. In accordance with one embodiment a method of treating a patient with an opioid addition is provided wherein the patient is administered a pharmaceutical composition comprising a PSD95-nNOS inhibitor, wherein the PSD95-nNOS inhibitor is

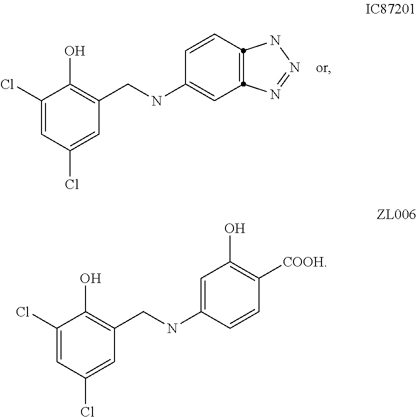

In one embodiment described herein is a novel animal model for studying a response to at least one opioid in a patient wherein the patient has neuropathic pain, and wherein the response is a perseveration of responding. In another embodiment, the disclosure provides an nNOS-PSD95 disruptor compound, wherein the molecule inhibits opioid-induced dopamine efflux in a neural cell and wherein the molecule does not itself produce a reward response in a the neural cell.

In one embodiment, an animal model system for studying a response to at least one opioid in a patient is provided. The model system comprises a rodent species surgically modified to allow self-administration of an opioid either in the presence or absence of a compound that disrupts the protein-protein interface between nNOS and PSD95. In one embodiment, the animal model system is optimized to represent a patient having neuropathic pain and wherein the response is a preservation of responding. In one embodiment the animal model system comprises a compound that disrupts the protein-protein interface between nNOS and PSD95, wherein the compound comprises the general structure of

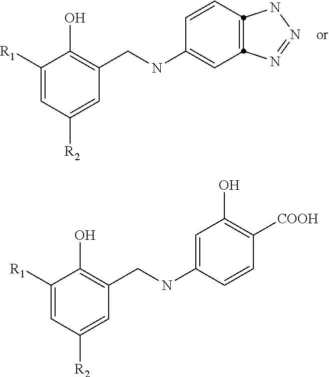

wherein R₁ and R₂ are independently selected from F, Br and Cl, optionally wherein R₁ and R₂ are each Cl.

In one embodiment, a composition for treating neurophathic pain is provided, wherein the composition comprises an opioid, a compound that disrupts the protein-protein interface between nNOS and PSD95, and a pharmaceutically acceptable carrier. In one embodiment the opioid is a compound selected from the group consisting of oxycodone, oxymorphone, hydromorphone, hydrocodone, morphine, tapentadol, tramadol, buprenorphine, and the physiologically acceptable salts thereof, and combinations thereof. In a further embodiment the compound that disrupts the protein-protein interface between nNOS and PSD95 is a compound comprising the general structure of

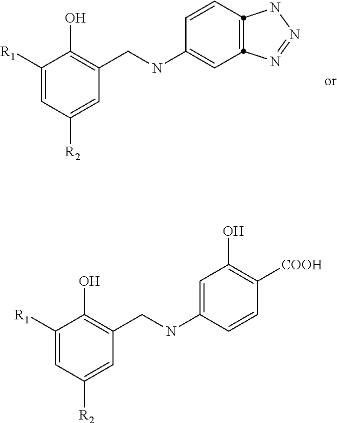

wherein R₁ and R₂ are independently selected from F, Br and Cl, optionally wherein R₁ and R₂ are each Cl.

In another embodiment, a method is provided for inhibiting opioid-induced dopamine efflux in a neural cell comprising administering to a patient a nNOS-PSD95 disruptor molecule. In an alternative embodiment, a method is provided for blocking opioid-reward responses in a neural cell comprising administering to a patient a nNOS-PSD95 disruptor molecule.

IC87201, the first-in-class small molecule inhibitor of PSD95-nNOS interaction, was previously disclosed by applicants. As reported herein IC87201 interrupts NMDAR-dependent nNOS signaling and suppresses pathological pain in animal models without altering basal nociceptive thresholds in the absence of injury (Br J Pharmacol. (2009); 158(2): p 494-506). Importantly, as reported herein IC87201 and a related analog, ZL006 (Nat Med (2010); 16(12), p. 1439-1443), block development of morphine reward in a conditioned place preference assay without producing memory impairment (see Example 1). Thus PSD95-nNOS inhibitors lack many unwanted side-effects of NMDAR antagonists.

### Example 1

By exploiting the specificity of NMDAR-Nnos signaling, we expect to block opioid-induced reward and DA efflux while sparing mu opioid receptor (MOR) mediated therapeutic efficacy. We will test our hypothesis by completing experiments under two Specific Aims:

Aim 1: To test the hypothesis that disruption of PSD95-nNOS protein-protein interactions will suppress morphine-induced reward. We will use conditioned place preference (CPP) to test the hypothesis that PSD95-nNOS disruption attenuates opioid reward (defined by a rightward shift in the dose-response curve for morphine to elicit CPP). A NO donor will be used to counteract this effect. Paw withdrawal thresholds will be assessed before and after drug pairings to identify the impact of PSD95-nNOS disruption on morphine antinociception. We will also use drug self-administration to determine whether PSD95-nNOS inhibitors (IC87201, ZL006) suppress acquisition and/or maintenance of morphine self-administration. In all studies, comparisons will be made with an inactive analog (ZL007) that does not disrupt PSD95-nNOS interactions.

Aim 2: To test the hypothesis that disruption of PSD95-nNOS protein-protein interactions will attenuate opioid-induced DA dynamics in the NAc shell. Fast-scan cyclic voltammetry (FSCV) will be used in conjunction with stimulation of the medial forebrain bundle (MPH) to promote DA efflux in NAc shell in vivo. Our experience with this technology permits real-time assessments of evoked DA release and uptake while testing the ability of IC87201 and ZL006 to alter DA dynamics both alone and in conjunction with morphine. We anticipate an attenuation of DA by PSD95-nNOS inhibitors but not ZL007 based upon our preliminary studies.

Our results validate our innovative approach of disrupting protein-protein interactions to reconfigure neuronal signaling to suppress opioid reward and DA efflux—eliminating aberrant signaling cascades involved in initiation of drug seeking, while preserving mechanisms required for normal physiological functions. Our project will also validate the innovative approach of decoupling NMDAR-dependent nNOS signaling as a previously unrecognized therapeutic strategy for reducing opioid abuse liability. Most importantly, validation of our hypotheses will promote the parallel benefit of developing second-generation PSD95-nNOS inhibitors aimed at preventing and treating prescription opioid abuse, thereby closing a major gap in an area of unmet therapeutic need.

Approach: Our approach is to specifically disrupt the protein-protein interaction between PSD95 and nNOS as a therapeutic strategy to inhibit NMDAR-dependent NO signaling and attenuate opioid reward. NMDAR antagonists (e.g. MK801) inhibit CPP to morphine as well as morphine tolerance and withdrawal but adverse side effects (ataxia, memory impairment, psychotomimetic effects, enhanced morphine lethality) and low therapeutic indices limit their clinical use. Most nNOS catalytic inhibitors are not selective, increase blood pressure and are unsuitable for chronic use.

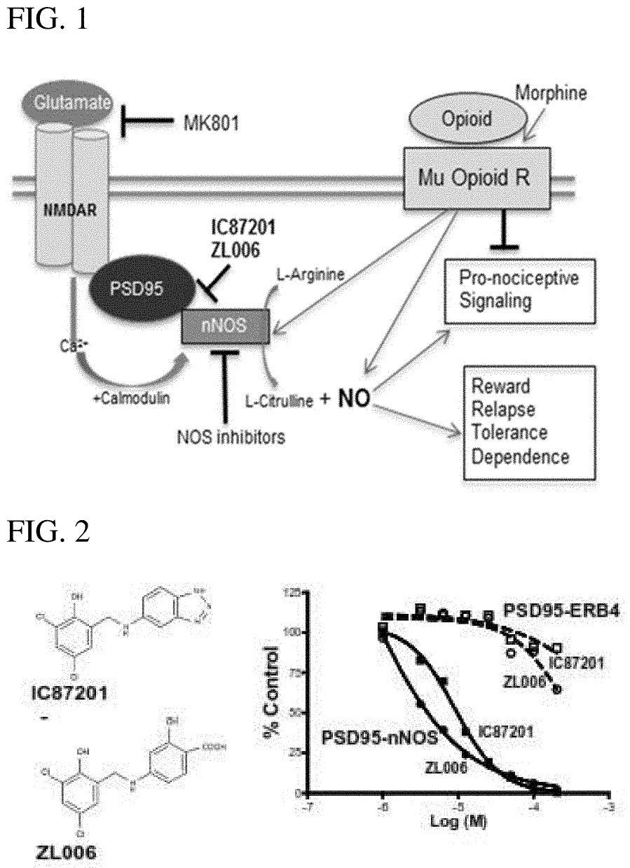

Mechanism: We developed an in vitro assay using Alphascreen to establish whether IC87201 and ZL006 directly disrupt interactions between purified nNOS and PSD95 proteins. IC87201 and ZL006 inhibited PSD95-nNOS binding in vitro without disrupting interaction of PSD95 with unrelated proteins. IC87201 was first identified in a High Throughput Screen and shown to disrupt PSD95-nNOS binding. A later group identified ZL006. ZL006, but not the inactive analog ZL007, inhibits ischemia induced damage in wild type (WT) but not in nNOS −/− mice. The active analog also inhibits the association of nNOS with PSD95 in ischemic cortices of WT but not nNOS −/−mice. The active analog also inhibits the association of nNOS with PSD95 in ischemic cortices of −/− mice. Our studies provide the first demonstration that ZL006 disrupts PSD95-nNOS interactions through a direct mechanism (FIG. 2). We also verified that IC87201 and ZL006 inhibit NMDA-induced NO formation (data not shown Smith et al. (2016) Behavioural Brain Research 305: 23-29); NO formation increases intracellular cGMP levels via activation of guanylate cyclase. IC87201 and ZL006 suppressed NMDA-stimulated cGMP levels in primary hippocampal cultures similar to MK801 (Hohmann lab, published in Smith et al. (2016) Behavioural Brain Research 305: 23-29). Thus, these small molecules disrupt NMDAR-dependent NO signaling in vitro and in vivo.

For in vivo proof of concept studies, we test these compounds using methods that are established in our labs and validated in the literature for use in assessing: 1) the reinforcing properties of drugs (CPP and drug self-administration) and 2) real-time DA dynamics with FSCV. For controls, we will use the inactive analog ZL007 to show dissociation between effects of active and inactive inhibitors. All studies use n=10 rats per group.

### Example 2: Disruption of PSD95-nNOS Protein-Protein Interactions Will Suppress Morphine-Induced Reward

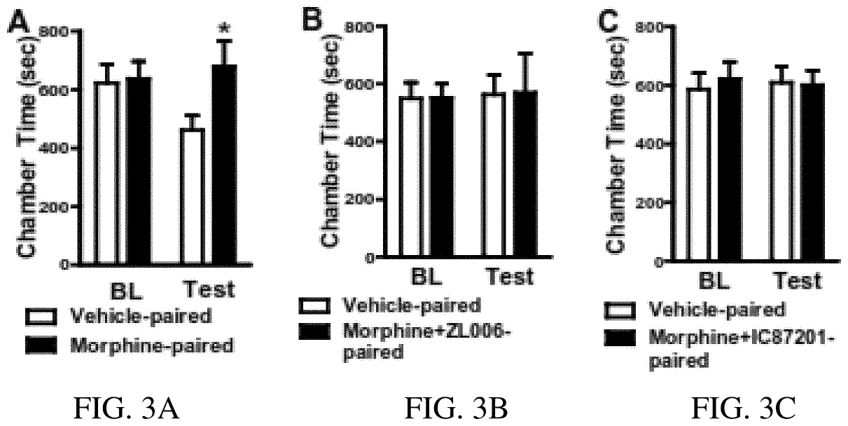

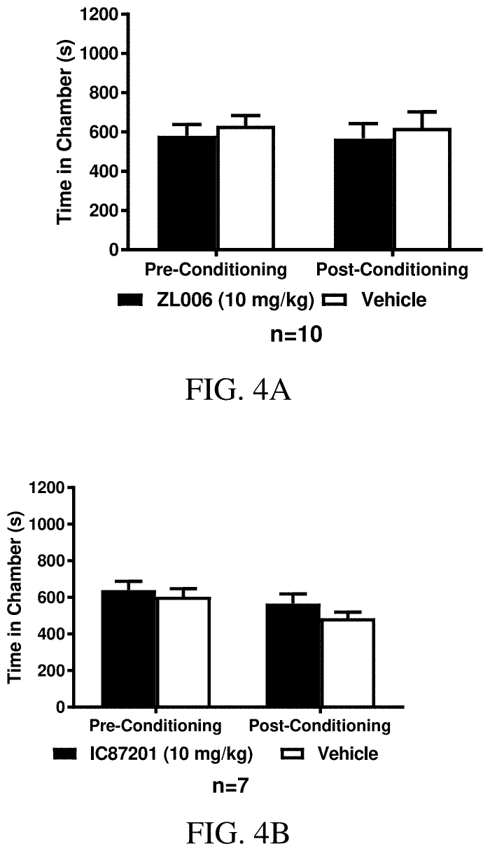

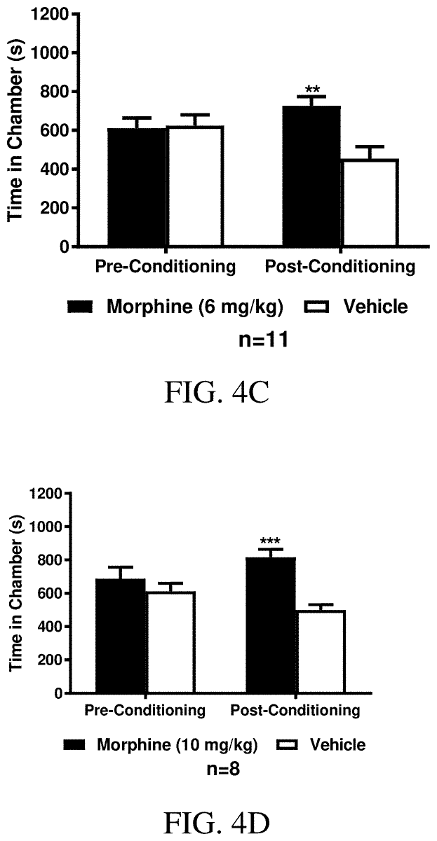

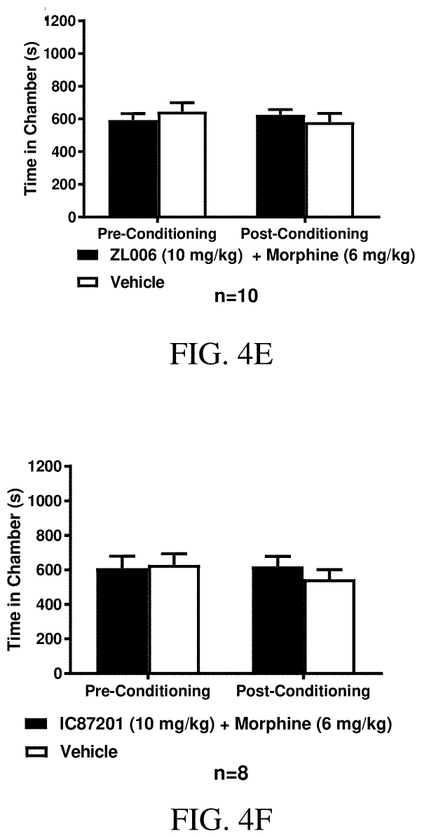

Overview: Morphine-induced CPP is blocked in rats by the NMDAR antagonist MK801 (0.1 mg/kg i.p.) at doses that do not alter chamber preference. Increasing NO production enhances both acquisition and expression of CPP to morphine. The NO donor L-arginine (in the central amygdala) increased acquisition of CPP to morphine and this enhancement was blocked by the NOS inhibitor L-NAME. Our preliminary studies verify that naïve animals exhibit robust CPP to morphine (FIG. 3A). However, morphine-induced CPP was notably absent when the opioid was co-administered with either ZL006 or IC87201 (FIGS. 3B and 3C). The same results are observed with repeated pairings of the treatments (FIGS. 4A-F), supporting our contention that PSD95-nNOS inhibitors suppress opioid reward.

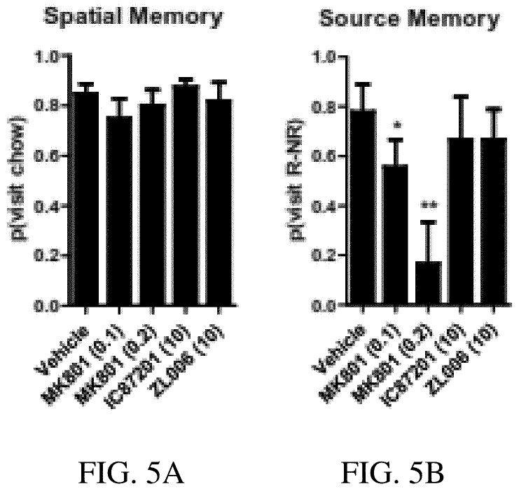

Memory impairment can confound CPP studies that depend on learning. Experiments were conducted to determine whether PSD95-nNOS inhibitors impair normal memory functions or show favorable side effect profiles compared to NMDAR antagonist MK801. We used a highly sensitive “source memory” task developed by applicants for use in rats to better mimic types of memory that are actually impaired in humans. Memory for source (i.e., origin) of information was eliminated (i.e. did not differ from chance) in MK801-treated rats under conditions in which spatial working memory (assessed concurrently in the radial maze) was unaffected. By contrast, IC87201 and ZL006 failed to impair spatial memory or source memory under identical conditions. Moreover, rats showed no decreases in accuracy in visiting chow locations in the maze, indicating that motor impairing side effects that could otherwise confound assessment of radial arm maze performance were absent. High accuracy in revisiting chow locations also demonstrates that PSD95-nNOS disruptors did not impede food motivated behavior; there was no difference between groups receiving vehicle or PSD95-nNOS disruptors in motivation to visit chow locations. Moreover, IC87201 and ZL006 did not impair memory (FIG. 5A) in the Morris Water maze or novel object recognition test (data not show) or impair motor performance in the rota-rod test (10-30 mg/kg i.p. (FIG. 5B)). This class of molecules, therefore, exhibits an improved safety profile compared to NMDAR antagonists: Hypothesis 1: Active (ZL006) but not inactive (ZL007) PSD95-nNOS disruptors will block CPP to morphine in an NO-dependent manner PSD95-nNOS disruptors will not by themselves produce reward.

### Example 3 PSD95-nNOS Disruption Shifts the Dose Response Curve for Morphine to Produce CPP

We will characterize the dose response relationship for morphine to produce CPP in the presence and absence of active (ZL006) and inactive (ZL007) PSD95-nNOS inhibitors and vehicle. We will use the NO donor molsidomine (30 min i.p. pretreatment, at doses that do not alter chamber preference, to surmount effects of PSD95-nNOS disruption. We will measure, in duplicate in each paw, mechanical paw withdrawal thresholds (evoked on the plantar paw surface through the apparatus grid floor) after each drug pairing using methods established in our lab. These latter studies will determine any impact of PSD95-nNOS disruption on morphine antinociception. Note that CPP to morphine is always determined in the drug free state. Studies will employ doses of active compounds that do not impair memory or motor functions.

Method: On CPP day 1 and 2, rats are allowed to habituate to the apparatus. On CPP day 3, rats are again allowed to explore the apparatus (30 min) to verify absence of chamber bias. Only rats that fail to show a chamber bias are used for CPP experiments. Rats used for CPP are injected (i.p.) with test drug (day 4, 6 and 8) prior to placement in the drug-paired chamber. On alternate days (day 5, 7 and 9), rats receive vehicle (i.p.) and are placed in the opposite chamber. Mechanical paw withdrawal thresholds to an electronic von Frey anesthesiometer are measured in duplicate through the grid floor of our custom apparatus before and after each drug pairings to assess possible changes in nociceptive thresholds. Motor behavior is tracked by photobeams. CPP is measured (day 10) in the drug-free state. Three doses of morphine are evaluated so that the dose response curve for morphine to produce CPP can be determined in the presence and absence of active and inactive PSD95-nNOS inhibitors. One morphine dose is tested per group. These studies require 19 groups receiving drug-vehicle pairings where drug (i.p.) refers to: 1) vehicle; 2) ZL006 [10 mg/kg]; 3) ZL007 [10 mg/kg]; 4-6) morphine [3 doses; e.g. 3, 6, 10 mg/kg]; 7-9) morphine [3 doses]+ZL006; 10-12) morphine [3 doses]+ZL007; 13) molsidomine [10 mg/kg]; 14-16) morphine [3 doses]+molsidomine; 17-19) morphine [3 doses]+molsidomine+ZL006. N=190.

Anticipated Results: Active (ZL006) but not inactive (ZL007) analogs will produce rightward shifts in the dose response curve for morphine to produce CPP. The NO donor molsidomine will surmount effects of ZL006 on morphine CPP. PSD95-nNOS disruption will not impede (and may enhance) acute morphine antinociception (as measured by morphine-induced changes in mechanical paw withdrawal thresholds). ZL006 will not produce either CPP or aversion in the absence of morphine.

We test mechanical thresholds in same rats used for CPP. PSD95-nNOS inhibitors did not alter tail flick latencies (i.e. produce antinociception) in naïve animals. Future studies will assess impact of these agents on morphine antinociceptive tolerance. Memory impairment can't confound our interpretation of robust single trial CPP to morphine; acute PSD95-nNOS disruptors (i.p.) did not impair memory (FIG. 5A; data not shown) or motor function. We are also prepared to repeat experiments evaluating memory and motor function using the same injection schedule used for CPP. We also expect to see a dissociation between effects of active and inactive analogs—(run under identical and blinded conditions) on morphine CPP; thus, nonspecific disruptions of conditioning are unlikely to confound data interpretation. Moreover, CPP is always evaluated in the drug-free state. Finally, we will examine the impact of PSD95-nNOS inhibitors on motivation to self-administer morphine.

### Example 4: PSD95-nNOS Disruptors Will Suppress Acquisition and Maintenance of Morphine Self-Administration

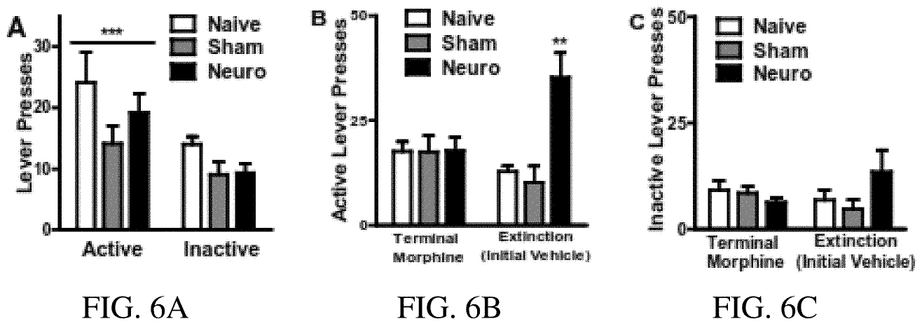

Overview: A two lever drug self-administration approach will be used to complement results obtained using CPP. This method is used in our published and preliminary studies (FIGS. 6A-6C).

We compared acquisition, maintenance and extinction of morphine self-administration in the presence and absence of a pathological pain state (see FIGS. 6A-6C). These preliminary studies were not designed to study opioid dependence but, rather, to determine whether neuropathic animals responded differentially from naïve and sham animals during extinction; a low morphine dose was used here to obtain conditions in which absolute levels of morphine self administration were similar between naïve, neuropathic and sham groups prior to the extinction test.

We also used self-administration to model therapeutic self-medication and compare the impact of chronic pain on the maintenance, extinction and reinstatement of self-administration with a cannabinoid CB2 Analgesic. Most pertinent to the proposed studies, NMDAR antagonists block acquisition of morphine self-administration and morphine self-administration is modulated by NO; acute and chronic effects of NO precursor L-arginine (0.1 mg/kg i.p.) on morphine self-administration are blocked by NOS inhibitor L-NAME (5-15 mg/kg i.p.). These studies establish feasibility of using drug self-administration to assess impact of nNOS-targeting strategies on acquisition and maintenance of morphine reward.

General Methods: Rats are food restricted, pretrained to lever press for food on both levers and then returned to ad lib food one week before surgery. Only rats that use both levers equally are used for self-administration. After surgical recovery and reestablishment of lever training, rats are switched.

We will examine the impact of PSD95-nNOS disruptors on i.v. morphine (0.75 mg/kg/infusion) self-administration over 12 consecutive sessions under fixed ratio 1 (FR1) schedule of reinforcement. In separate studies, the PSD95nNOS inhibitor (or inactive analog) is given during either: i) acquisition (e.g. session 1-6; Study #2), or ii) maintenance (e.g. session 7-12; Study #3), of morphine self-administration. Pressing the active (left or right, counterbalanced) but not inactive lever elicits an infusion. We will determine the impact of pretreatment with active PSD95-nNOS inhibitors (10 mg/kg i.p.), an inactive analog or vehicle on morphine self-administration. Drugs are given i.p. 30 min prior to lever access. Self-administration sessions are limited to 50 min (used in FIGS. 6A-6C). Our pharmacokinetic studies show therapeutic drug levels in blood are sustained for this entire duration following i.p. administration (data not shown). Active and inactive lever responses are assessed.

### Example 5: Do PSD95-nNOS Inhibitors Suppress the Acquisition and/or Maintenance of Morphine Self-Administration?

Active PSD95-nNOS inhibitors (IC87201, ZL006), an inactive analog (ZL007) or vehicle is given i.p. 30 min prior to lever access for all self-administration sessions corresponding to the targeted manipulation phase: acquisition or maintenance. Doses (i.p.) used are those shown to block morphine CPP but may be adjusted as required. Rats are allowed to self-administer morphine (0.75 mg/kg/bolus i.v.) under FR1 schedule.

This study requires 4 i.p. drug groups (vehicle, IC87201, ZL006, ZL007)×2 self-administration (i.v.) conditions (morphine, saline)×2 i.p. drug manipulation phases (Study #2: acquisition (e.g. session 1-6); Study #3: maintenance (e.g. session 7-12)×10 rats per group. N=160.

Anticipated Results: Active (IC87201, ZL006) PSD95-nNOS inhibitors but not inactive (ZL007) analogs will impair both acquisition and maintenance of morphine self-administration. IC87201 and ZL006 will suppress responding on the active (morphine-paired), but not the inactive, lever.

Inactive lever responding will be monitored and mechanical paw withdrawal thresholds assessed before and after each self-administration session. We can space sessions if necessary.

Preferential responding on the active (vs. inactive) lever demonstrates specificity of self-administration. Rats are pre-trained to lever press for food on both levers and are never food restricted following completion of lever training. Rats exhibiting an initial lever bias are not used. We are also prepared to verify whether doses of PSD95-nNOS inhibitors that block morphine self-administration impede lever pressing for food under analogous conditions. However, this possibility is unlikely because PSD95-nNOS inhibitors did not impede food-motivated behavior in our radial arm maze task.

Our PK studies show that drug levels of inhibitors are elevated and stable for the duration of the 50 min session. We can increase effort required by rats to obtain drug using FR5 schedule rather than increasing session length. We also use a morphine dose validated in self-administration literature.

PSD95-nNOS inhibitors blocked CPP to morphine. We can evaluate extinction and reinstatement of morphine self-administration using our published methods.

### Example 6: To Test the Hypothesis that Disruption of PSD95-nNOS Protein-Protein Interactions Will Attenuate Opioid-Induced DA Dynamics in the NAc Shell

Overview: Drugs of abuse increase DA transmission most prominently in the NAc, a forebrain region crucial for translating motivational input into motor output. DA neurons innervating the NAc shell, which are implicated in motivational effects of abused drugs, are preferentially impacted. In fact, opioid self-administration increases activity of mesolimbic DA neurons immediately before the next lever-press, highlighting DA involvement in the drive for drug taking. Although other mechanisms, including changes in glutamate plasticity, come into play, an increase in NAc DA is crucial for the transition to addiction. Here, we use FSCV in NAc shell in combination with electrical stimulation of the MFB to record the impact of PSD95-nNOS disruption on real-time changes in evoked DA release and on the DA response to morphine.

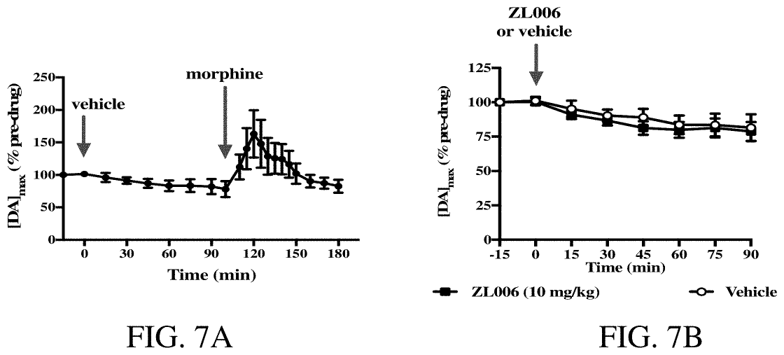

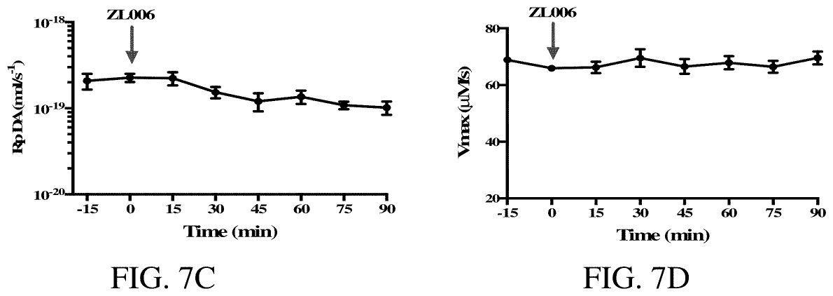

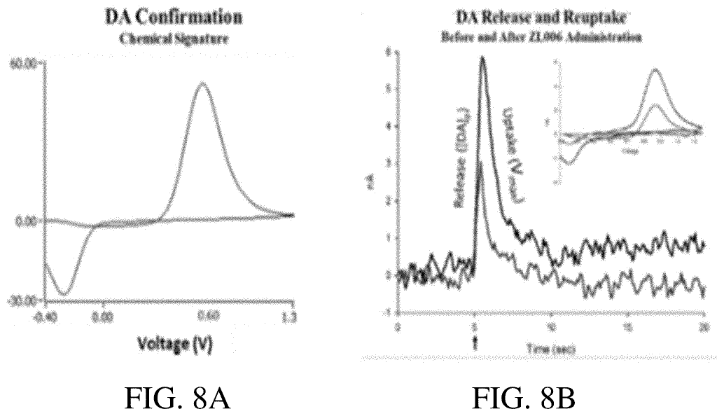

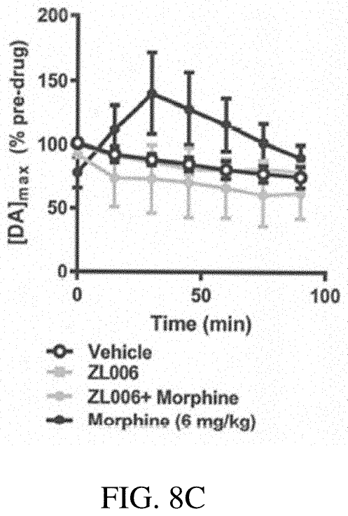

Design and Rationale: Although FSCV can record spontaneously occurring DA transients in behaving animals, the anesthetized preparation is preferred because it provides reliable and reproducible DA signals independent of behavioral activity. In fact, the fluctuating and intermittent nature of behavior-related DA signals (e.g.,) could confound efforts to establish a first-ever characterization of PSD95-nNOS disrupters on DA dynamics. We use urethane (1.5 mg/kg) anesthesia because it does not alter DA uptake kinetics. Rats are fixed in a stereotaxic frame, and a carbon-fiber working electrode (6 μm diameter) is lowered into NAc shell. A bipolar stimulating electrode is lowered over the ipsilateral MFB to evoke endogenous DA release. An Ag/AgCl reference is placed on the contralateral cortex. A bipotentiostat controlled by TH-1 Software (ESA, Chelmsford, Mass., USA) applies a triangular waveform (−0.4 V to +1.3 V and back) to the working electrode, which allows for DA detection: DA oxidizes at approximately +0.6 V and reduces at −0.3 V. As we and others have shown, these oxidation and reduction peaks create a unique chemical signature (voltammogram) for DA (FIG. 7A). The DA voltammogram is readily distinguishable from potential interferant molecules, including our test compounds, which are first monitored in a flow cell to rule out electrochemical interference. Voltammograms are obtained every 100 ms at a scan rate of 400 v/s.

### Example 7: Does PSD95-nNOS Disruption Attenuate the Evoked DA Signal in NAc Shell?

The basic design is to collect baseline DA signals (30 min) evoked by MFB stimulation, administer (ip) test drug(s) or vehicle, and collect post-administration evoked DA signals. Analysis will be conducted with and without morphine. We will evaluate dose-dependent effects (1, 4, and 10 mg/kg) of IC87201 and ZL006 and test 10 mg/kg of the inactive analog (ZL007) and vehicle. The effects of each compound (injected i.p.) will be monitored for at least 60 min and tested in separate groups of rats (n=10/group). In our morphine experiments, we will combine a ZL006 dose (selected from Study #4), ZL007, or vehicle with 6 mg/kg morphine. To evoke DA release, we apply a biphasic current (60 Hz, 0.4 s, 300 μA) to the stimulating electrode. Although the DA signal increases with increasing stimulation frequency over a range of 10-100 Hz, we use 60 Hz because it elicits DA concentrations >1 μM, which exceeds DA uptake Km (0.2 μM) and ensures accurate determination of Vmax. Our pilot data show a clear decrease in DA signal within 30 min after ZL006 (FIG. 7B).

Stimulations at 60 Hz are applied every 2 min to ensure renewal of the readily releasable DA pool. Evoked signals are resolved into respective rates for release and uptake, and pre- and postdrug data are compared. Upon completion of recording, the working electrode is used for postcalibration testing to estimate the in vivo concentration of DA. The analysis used to assess changes in DA release and uptake involves a modification of Michaelis-Menten kinetics. We use mathematical modeling that describes the rate of change in evoked DA concentration as the counteracting balance of release and reuptake: d[DA]/dt=[DA]p*f−Vmax/(Km/[DA]+1), where [DA] is the extracellular DA concentration, [DA]p refers to the extracellular DA concentration evoked per stimulus pulse, Vmax represents the Michaelis-Menten term for maximal uptake rate, Km is the Michaelis-Menten term inversely proportional to transporter affinity, and f is the frequency of the stimulation train. Thus, both Km and Vmax are Michaelis-Menten rate constants that describe uptake (see FIG. 7B). [DA]p describes the rate of stimulated release. Km is held constant at a 0.2 μM, the reported value in striatum for anesthetized rodents. [DA]p and Vmax are determined by the above equation in combination with curve-fitting software that incorporates a simplex minimization algorithm.

Our studies show ZL006 does not increase DA release and attenuates opioid-induced DA efflux induced by morphine in the nucleus accumbens shell. Active PSD95-nNOS inhibitors (ZL006) did not enhance evoked DA release and blocked the morphine-induced increase in evoked DA in NAc shell.

While anesthetics could potentially change the pharmacokinetics of disruptors, urethane anesthesia does not alter DA dynamics. Pentobarbital, which does not interfere with DA transport activity, could be used as an alternative. 60 Hz stimulation frequencies are reliably used to assess drug-induced changes in DA dynamics minimizing that chance of possible effects specific to DA firing rate or pattern. We will also test in the 20-40 Hz range because DA neurons can switch between tonic and burst firing. Although early evidence suggested that DA neurons fire maximally at 20 spikes/s, sustained DA release is observed at frequencies up to 100 Hz and putative DA neurons exhibit high firing rates in vivo. The DA signal is distinct from potential interferants, and our test drugs are characterized in a flow cell before in vivo testing.

### Experimental Details

The experiments disclosed herein are conducted to confirm that disruption of PSD95-nNOS protein-protein interactions will suppress opioid reward using well-established preclinical models. Only male rats were used because the literature suggesting that NMDAR antagonists and NOS inhibitors disrupt opioid reward was all collected in male rats; thus initial validation of our phenomenon in male rats is necessary to prevent replication of the literature in female rats prior to undertaking the present work. Pending validation of our hypotheses, possible sex differences will be evaluated in future work. Animals will be used to complete six separate experiments under three Specific Aims as follows:

(Aim 1)

Rats are used in the Multi-Science II Building of Indiana University. All experiments will be conducted following approval by Indiana University Bloomington (IUB) IACUC committee. Studies are conducted to confirm that disruption of PSD95-nNOS protein-protein interactions will suppress morphine-induced reward.

Study #1: Does PSD95-nNOS disruption shift the dose response curve for morphine to produce CPP?

This study requires 19 groups of rats evaluated using a conditioned place preference approach. In the same animals we measure responsiveness to punctuate mechanical stimulation with an electrovonfrey anesthesiometer (Rahn et al. (2014) Molecular Pain 2014, 10:27 DOI: 10.1186/1744-8069-10-27) before and after all drug or vehicle pairings. Animals are able to freely move and withdraw from this stimulation (delivered through the grid floor of the CPP apparatus); this dependent measure permits assessment of impact of test drugs on morphine antinociception in the same animals used for CPP and is, therefore, consistent with our obligations to conserve unnecessary use of animals. An NO donor (molsidomine) is used in an attempt to surmount impact of PSD95-nNOS inhibitor on CPP to morphine. Compounds are tested under blinded conditions. On the days of conditioning, animals receive the following drug-vehicle pairings where drug refers to: 1) vehicle; 2) ZL006 (10 mg/kg); 3) ZL007 (10 mg/kg); 4-6) morphine [3 doses; e.g. 3, 6, 10 mg/kg]; 79) morphine [3 doses]+ZL006; 10-12) morphine [3 doses]+ZL007; 13) molsidomine (10 mg/kg); 1416) morphine [3 doses]+molsidomine; 17-19) morphine [3 doses]+molsidomine+ZL006. N=190: This number reflects testing of 19 cohorts of 10 rats.

Study #2: Do PSD95-nNOS inhibitors suppress the acquisition of morphine self-administration?

General animal issues for all drug self-administration studies: Animals are surgically implanted with jugular catheters for intravenous drug self-administration (Study #2-3). All surgeons are trained in aseptic surgery. Catheters are flushed daily to prevent occlusion of catheters and minimize subject losses due to occasional occlusion of catheters. Sample sizes of 10 rats per group are used to ensure sufficient power to detect group changes in lever pressing behavior in the event of a 10% loss of subjects due to catheter occlusion based upon previous studies of similar duration. Animals are allowed to self-administer morphine or saline for 12 consecutive days. Paw withdrawal thresholds are always assessed before and after each self-administration session to further ensure that carryover drug effects are absent prior to initiating the next self-administration session. Monitoring this dependent measure allows us to space sessions as required, which helps us constrain unnecessary use of animals.

In this study, PSD95-nNOS inhibitors, an inactive analog or vehicle is administered to rats only during acquisition of morphine self-administration (e.g. session 1-6). Completion of Study #2 requires the following 16 treatment groups: 2 self-administration (i.v.) conditions (morphine, saline)×4 drug (i.p.) pretreatments [active nNOS-PSD95 inhibitors (ZL006, IC87201); inactive nNOS-PSD95 inhibitor (ZL007)]; one vehicle]×10 animals per group Study #2: N=80

Study #3: Do PSD95-nNOS inhibitors suppress the maintenance of morphine self-administration?

In this study, nNOS-PSD95 inhibitors, their inactive analogs or vehicle is administered to rats only during maintenance of morphine self-administration (e.g. session 7-12). Animals are allowed to self-administer morphine over 12 consecutive days. Completion of Study #3 requires the following five treatment groups: 2 self-administration (i.v.) conditions (morphine, saline)×4 drug (i.p.) pretreatments [active nNOS-PSD95 inhibitors (ZL006, IC87201); inactive nNOS-PSD95 inhibitor (ZL007)]; one vehicle]×10 animals per group Study #3: N=80.

(Aim 2)

Testing the hypothesis that disruption of PSD95-nNOS protein-protein interactions will attenuate opioid-induced DA dynamics in the NAc shell.

Study #4-5: Does PSD95-nNOS disruption attenuate the evoked DA signal in NAc shell?

Male Sprague-Dawley rats (2-4 months of age) are obtained from Harlan Industries (Indianapolis, Ind.). Animals are single-housed in a temperature and humidity controlled vivarium, with ad-libitum access to food and water. All housing and experimental procedures are approved by the Institutional Animal Care and Use Committee (IACUC). We anticipate using 60 animals for testing novel small molecule inhibitors of nNOS-PSD95 protein-protein interactions on DA dynamics: 30 rats for ZL006 (10 at each of 3 doses); 10 rats for ZL007; 10 rats for vehicle; and 30 rats for IC87201 (10 at each of 3 doses). In addition, we will use 30 rats for testing manipulations of morphine-induced changes in DA dynamics: 10 rats for morphine+ZL006; 10 rats for morphine+ZL007; and 10 rats for morphine+vehicle. Together, our animal numbers are in keeping with past research indicating the numbers necessary for obtaining rich FSCV datasets. Proposed doses are based on a well-established literature for morphine and on our ongoing studies with PSD95nNOS disruptors. Total animal use in Aim 2 is 110 rats.

### Justification for the Use of Animals

(Aim 1)

Rodents are used because they are the lowest form of life for which there is an adequate base of knowledge to perform the proposed experiments. Models of opiate reward, tolerance, dependence and relapse are well validate in rats.

A large literature exists on the physiology and neurochemistry of nociception and drug abuse in rats. Rodents are appropriate for the proposed research because progress toward understanding the biological basis of opiate addiction requires that procedures are conducted with animals.

Rats are the most appropriate species for the proposed studies research because:

(a) there is extensive information about the behavior of rats in related research and the use of conditioned place preference and drug self-administration to assess drug reward is well-established in this species. These factors reduce the need to determine the feasibility of conducting the proposed experiments in other species (e.g. transgenic mouse lines); (b) conditioned place preference, and drug self-administration and facilities as well as equipment for assessing and monitoring antinociception (i.e. mechanical paw withdrawal thresholds) are currently available for testing with rats in our labs; (c) the PIs and research team has extensive experience with all components of the proposed methods in the proposed species; and (d) there is extensive information about the neural basis of drug abuse and antinociception in rats.

(Aim 2)

Understanding the potential translational value of PSD95-nNOS protein-protein interactions with the DA system requires in vivo testing. Animal models permit the use of invasive recording techniques that are neither feasible nor ethically appropriate in humans. Such techniques provide direct access to physiological data that cannot be resolved with sufficient temporal or spatial resolution for our purposes except by the use of implanted electrodes. We plan to use a total of 110 rats (see above).

What is claimed is:

1. A method of inhibiting opioid-induced dopamine efflux in a neural cell, said method comprising contacting said cell with a compound that disrupts the protein-protein interface between nNOS and PSD95.

2. The method of claim 1 wherein said compound comprises the general structure of Formula I: 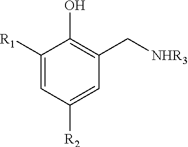
wherein R₁ and R₂ are independently selected from the group consisting of I, F, Br and Cl;
and R₃ is

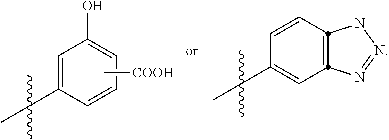

3. The method of claim 2 wherein said compound comprises the general structure of 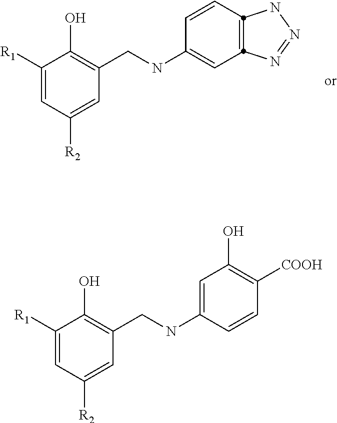
wherein R₁ and R₂ are independently selected from F, Br and Cl.

4. The method of claim 3 wherein R₁ and R₂ are each Cl.

5. A method of treating a patient with an opioid addiction, said method comprising the steps of administering a pharmaceutical composition comprising a compound that disrupts the protein-protein interface between nNOS and PSD95.

6. The method of claim 5 wherein said compound comprises the general structure of Formula I: 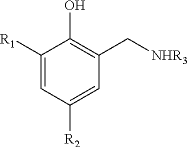
wherein R₁ and R₂ are independently selected from the group consisting of I, F, Br and Cl;
and R₃ is

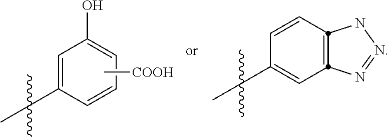

7. The method of claim 6 wherein said compound comprises the general structure of 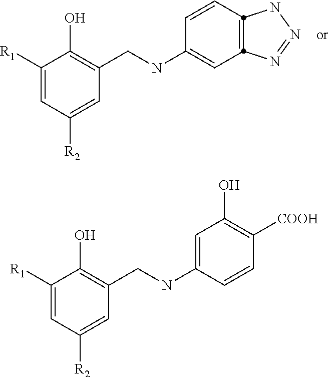
wherein R₁ and R₂ are independently selected from F, Br and Cl.

8. The method of claim 7 wherein R₁ and R₂ are each Cl.

9. A composition for treating neurophathic pain, said composition comprising an opioid;
a compound that disrupts the protein-protein interface between nNOS and PSD95; and
a pharmaceutically acceptable carrier.

10. The composition of claim 9 wherein said opioid is a compound selected from the group consisting of oxycodone, oxymorphone, hydromorphone, hydrocodone, morphine, tapentadol, tramadol, buprenorphine, and the physiologically acceptable salts thereof, and combinations thereof.

11. The composition of claim 10 wherein said compound comprises the general structure of 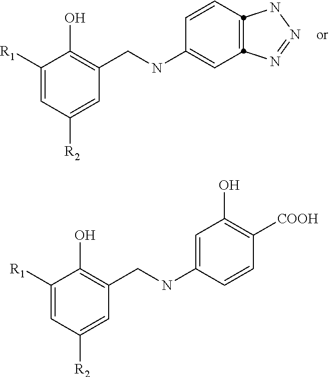
wherein R₁ and R₂ are independently selected from F, Br and Cl.

12. The method of claim 11 wherein R₁ and R₂ are each Cl.
